SEQUENCE LISTING (2 sequences)

SEQ ID NO: 1 - protein, 493 aa, Trichoderma reesei
MAGKLILVALASLVSLSIQQNCAALFGQCGGIGWSGTTCCVAGAQCSFVNDWYSQCLASTGGNPPNGTTSSSLVSRTSSASSSVGSSSPGGNSPTGSASTYTTTDTATVAPHSQSPYPSIAASSCGSWTLVDNVCCPSYCANDDTSESCSGCGTCTTPPSADCKSGTMYPEVHHVSSNESWHYSRSTHFGLTSGGACGFGLYGLCTKGSVTASWTDPMLGATCDAFCTAYPLLCKDPTGTTLRGNFAAPNGDYYTQFWSSLPGALDNYLSCGECIELIQTKPDGTDYAVGEAGYTDPITLEIVDSCPCSANSKWCCGPGADHCGEIDFKYGCPLPADSIHLDLSDIAMGRLQGNGSLTNGVIPTRYRRVQCPKVGNAYIWLRNGGGPYYFALTAVNTNGPGSVTKIEIKGADTDNWVALVHDPNYTSSRPQERYGSWVIPQGSGPFNLPVGIRLTSPTGEQIVNEQAIKTFTPPATGDPNFYYIDIGVQFSQN

SEQ ID NO: 2 - protein, 475 aa, Trichoderma reesei
QQNCAALFGQCGGIGWSGTTCCVAGAQCSFVNDWYSQCLASTGGNPPNGTTSSSLVSRTSSASSSVGSSSPGGNSPTGSASTYTTTDTATVAPHSQSPYPSIAASSCGSWTLVDNVCCPSYCANDDTSESCSGCGTCTTPPSADCKSGTMYPEVHHVSSNESWHYSRSTHFGLTSGGACGFGLYGLCTKGSVTASWTDPMLGATCDAFCTAYPLLCKDPTGTTLRGNFAAPNGDYYTQFWSSLPGALDNYLSCGECIELIQTKPDGTDYAVGEAGYTDPITLEIVDSCPCSANSKWCCGPGADHCGEIDFKYGCPLPADSIHLDLSDIAMGRLQGNGSLTNGVIPTRYRRVQCPKVGNAYIWLRNGGGPYYFALTAVNTNGPGSVTKIEIKGADTDNWVALVHDPNYTSSRPQERYGSWVIPQGSGPFNLPVGIRLTSPTGEQIVNEQAIKTFTPPATGDPNFYYIDIGVQFSQN

(19) United States
(12) Patent Application Publication
(10) Pub. No.: US 2011/0136182 A1
(43) Pub. Date: Jun. 9, 2011

(54) SWOLLENIN COMPOSITIONS AND METHODS OF INCREASING THE EFFICIENCY OF A CELLULASE

(75) Inventors: Tom Tao Huang, Fremont, CA (US); Colin Mitchinson, Half Moon Bay, CA (US); Rafael F. Sala, Mountain View, CA (US)

(73) Assignee: DANISCO US INC., Palo-Alto, CA (US)

(21) Appl. No.: 12/990,467

(22) PCT Filed: Apr. 29, 2009
(86) PCT No.: PCT/US2009/042102

§ 371 Date: Jan. 24, 2011

Related U.S. Application Data
(60) Provisional application No. 61/048,807, filed on Apr. 29, 2008.

(51) Int. Cl. C12P 19/14; C12N 9/96

(52) U.S. Cl. 435/99; 435/188

(57) ABSTRACT

Described are compositions and methods relating to enhancing the efficiency of cellulases for sugar production from cellulosic biomass using the polypeptide swollenin.

### PRIORITY

The present application claims priority to U.S. Provisional Application Ser. No. 61/048,807, filed on Apr. 29, 2008, which is hereby incorporated by reference.

### FIELD OF THE INVENTION

The compositions and methods relate to enhancing the efficiency of a cellulase in producing sugars from cellulosic biomass using the polypeptide swollenin.

### BACKGROUND

Interest in ethanol as a renewable fuel is stronger than ever before. The use of ethanol as a fuel additive has grown over the past few years and is expected to continue to grow in the foreseeable future. The use of ethanol reduces dependence on foreign oil, lowers greenhouse gas emissions, provides economic benefits for rural communities, and establishes a foundation for a biobased economy.

Potential feedstocks for cellulosic ethanol include corn stover, wheat straw, sugar cane bagasse, rice straw, paper pulp, wood chips, and biomass from “energy crops” such as fast-growing trees and grasses (switchgrass, prairie grass, miscanthus), dramatically expanding the available material for ethanol production. Although cellulosic biomass is available in large quantities, the main challenge for commercialization is to reduce the costs of an integrated biorefinery process that ultimately produced ethanol. Unlike starch, which contains homogenous and easily hydrolyzed polymers, typical cellulosic substrates/feedstocks for use in producing ethanol are not homogenous in nature. In addition to containing convertible cellulose and hemicellulose, they also contain lignin and other components, which cannot readily be converted to fermentable sugars.

Typically, raw biomass must be extensively pretreated chemically, physically, or biologically to produce a suitable substrate/feedstock for ethanol production. Physical pretreatment techniques include one or more of various types of milling, crushing, irradiation, steaming/steam explosion, and hydrothermolysis. Chemical pretreatment techniques include acid, alkaline, organic solvent, ammonia, sulfur dioxide, carbon dioxide, and pH-controlled hydrothermolysis.

There remains an urgent need to decrease the amount of hydrolytic enzyme necessary to convert the biomass to fermentable sugar and/or to decrease the amount of pretreatment necessary to make the biomass accessible to the hydrolytic enzymes.

### SUMMARY

In one aspect, a method for increasing the efficiency of a cellulase is provided, the method comprising (a) combining a cellulosic substrate, an amount of a whole cellulase and an amount of a swollenin and (b) incubating the cellulosic substrate, mixed cellulase composition and swollenin under conditions conducive to hydrolysis of cellulose. In a preferred implementation, the cellulosic substrate includes hydrogen-bonded molecules. The cellulosic substrate may be selected form the group consisting of wood, wood pulp, papermaking sludge, paper pulp waste streams, particle board, corn stover, corn fiber, rice, paper and pulp processing waste, woody or herbaceous plants, grasses, rice hulls, cottonstraw, corn cobs, distillers grains, leaves, wheat straw, coconut hair, switchgrass, and mixtures thereof.

In a related aspect, a method for increasing the efficiency of cellulose hydrolysis using a cellulase is provided, the method comprising: (a) combining a cellulosic substrate, a cellulase composition, and recombinant swollenin, and (b) incubating the cellulosic substrate, cellulase composition, and swollenin under conditions conducive to hydrolysis of cellulose, wherein the presence of recombinant swollenin increases the efficiency of cellulase hydrolysis by the cellulase composition compared to that obtained using a the cellulase composition in the absence of swollenin.

In some embodiments, the cellulase composition is a whole cellulase composition. In some embodiments, the cellulase composition is a mixed cellulase composition. In some embodiments, the cellulase composition comprises an endoglucanase, a cellobiohydrolases, and a β-glucosidase.

In some embodiments, the cellulase composition comprises one or more primary cellulases. In some embodiments, the cellulase composition consists essentially of one or more primary cellulases. In particular embodiments, the primary cellulases are selected from CBH1, CBH2, EG1, EG2, and β-glucosidase.

In some embodiments, the method is performed in the absence of accessory enzymes other than swollenin.

In some embodiments, the method is performed in the absence of EG4 and CIP1. In some embodiments, the method is performed in the absence of recombinant EG4 or recombinant CIP1. In some embodiments, the method is performed in the absence of recombinant EG4 and recombinant CIP1.

In some embodiments, the ratio of cellulases in the celluase composition to swollenin (wt:wt) is between about 20:1 and about 1:5. In some embodiments, the ratio of cellulases in the celluase composition to swollenin (wt:wt) is between about 10:1 and about 1:2. In some embodiments, the ratio of cellulases in the celluase composition to swollenin (wt:wt) is between about 5:1 and about 1:1.5.

In some embodiments, the swollenin and the cellulases are present in an approximately equal amount (wt:wt). Exemplary amounts of swollenin are from about 30% to about 70%, from about 40% to about 60%, and about 50%, of the enzymes used in the method (wt/wt).

In some embodiments, the cellulosic substrate is selected form the group consisting of wood, wood pulp, papermaking sludge, paper pulp waste streams, particle board, corn stover, corn fiber, rice, paper and pulp processing waste, woody or herbaceous plants, grasses, rice hulls, cottonstraw, corn cobs, distillers grains, leaves, wheat straw, coconut hair, switchgrass, and mixtures thereof. In some embodiments, the cellulosic substrate is a softwood. In some embodiments, the cellulosic substrate is high lignin substrate. In some embodiments, the cellulosic substrate has a kappa number of 80 or higher.

In some embodiments, the percent increase in cellulase efficiency is at least about 10%, at least about 15%, or even at least about 20%.

In another aspect, an enzyme composition is provided, comprising a mixed or whole cellulase composition and a swollenin. The ratio of mixed/whole cellulase to swollenin (wt:wt) may be between about 20:1 and about 1:5, inclusive. The ratio of mixed cellulase to swollenin (wt:wt) may further be between about 10:1 and about 1:2, inclusive. The ratio of mixed cellulase to swollenin (wt:wt) may even be between about 5:1 and about 1:1.5, inclusive.

In a related aspect, an enzyme composition is provided, comprising: (a) a mixed cellulase composition comprising an endoglucanase, a cellobiohydrolases, and a β-glucosidase, and (b) recombinant swollenin.

In some embodiments, the composition does not include EG4 or CIP1. In some embodiments, the composition does not include recombinant EG4 or recombinant CIP1. In some embodiments, the composition does not include recombinant EG4 and does not include recombinant CIP1.

In some embodiments, the mixed cellulase composition consists essentially of primary cellulases.

In another related aspect, an enzyme composition is provided, which consists essentially of: (a) a mixed cellulase composition comprising an endoglucanase, a cellobiohydrolases, and a 6-glucosidase, and (b) recombinant swollenin.

In some embodiments, the ratio of cellulases in any of the mixed cellulase compositions to swollenin (wt:wt) is between about 20:1 and about 1:5. In some embodiments, the ratio of cellulases in any of the mixed cellulase compositions to swollenin (wt:wt) is between about 10:1 and about 1:2. The composition of any of claims 20-25, wherein the ratio of cellulases in any of the mixed cellulase compositions to swollenin (wt:wt) is between about 5:1 and about 1:1.5. In some embodiments, the swollenin and the cellulases are present in an approximately equal amount (wt:wt).

In some embodiments, the amount of swollenin (wt:wt) in the composition replaces an approximately equal amount of cellulases (wt:wt) in the composition, with respect to cellulase efficiency on a cellulosic substrate.

These and other aspects of the compositions and methods will be apparent from the following description.

### BRIEF DESCRIPTION OF THE DRAWINGS

FIG. 1 illustrates glucose production by a mixed cellulase composition in the presence and absence of swollenin.

FIGS. 2A and 2B illustrate the effect of swollenin on cellulose hydrolysis of various cellulosic substrates.

FIG. 3 illustrates glucose production by 30 mg total enzyme per g cellulose with the enzyme being provided by various ratios of mixed cellulase to swollenin.

FIG. 4 illustrates percent cellulose digestion of softwood pulp at different relative concentrations of mixed cellulase and swollenin.

### DETAILED DESCRIPTION

### I. Definitions

Prior to describing the present compositions and methods, the following terms and phrases are defined. Terms not defined should be accorded their ordinary meaning as used in the art.

As used herein, “swollenin” refers to a protein/polypeptide that has the ability to facilitate weakening of filter paper and cause the swelling of cotton fibers without having cellulolytic activity, i.e., catalytic activity involving the breakage of individual cellulose strands into smaller monomer (glucose) or oligomers (polysaccharides). While it is useful to define swollenins loosely in terms of the expansin proteins described in McQueen-Mason et al. (1992) Plant Cell 4:1425-33, it is also apparent that microbial swollenins have distinct properties, for example, microbial swollenins are much larger proteins than plant expansins and have a low level of sequence identity with plant expansins. Moreover, certain microbial swollenin proteins exist in conjunction with a cellulose binding domain and may further exist in conjunction with a catalytic cellulase domain.

As used herein, the term “cellulosic substrate” or cellulosic feedstock” refers to materials which are composed of cellulose, hemi-cellulose, and β-glucans that are cross-linked with each other, and with lignin. Such cellulosic substrates may also contain other materials such as pectins, proteins starch and lipids, but preferably will have cellulose, hemi-cellulose and β-glucans as primary components.

As used herein, the terms “purification” and “isolation,” with reference to swollenin, refer to the separation of swollenin from some or all of the naturally occurring constituents with which it is associated in nature, or from some or all of the constituents with which it is associated following heterologous expression. The term “constituents” generally refers to other proteins, nucleic acids, lipids, cell wall material, and other cellular components.

As used herein, the term “kappa value” refers to the degree of lignification of a cellulosic substrate. Kappa values can be determined using, e.g., International Organization for Standardization document ISO 302:2004.

As used herein, the term “cellulose” refers a polysaccharide consisting of β(1→4) linked D-glucose units having the general formula (C₆H₁₀O₅)ₙ. Cellulose is the structural component of the primary cell wall of green plants, many forms of algae and the oomycetes.

As used herein, the term “cellulase” refers to an enzyme capable of hydrolyzing cellulose polymers to shorter oligomers and/or glucose.

As used herein, the term “whole cellulase composition/preparation/mixture” or the like refers to both naturally occurring and non-naturally occurring compositions that include a plurality of cellulases produced by an organism, for example a filamentous fungus. One example of a whole cellulase composition is medium (i.e., broth) in which filamentous fungi are cultured, which includes secreted cellulases, such as one or more cellobiohydrolases, one or more endoglucanases, and one or more β-glucosidases at a predetermined ratio.

As used herein, an “endoglucanase (EG)” is an enzyme (EC 3.2.1.4) that acts mainly on the amorphous parts of the cellulose fibre to hydrolyze internal β-1,4-glucosidic bonds in regions of low crystallinity.

As used herein, a “cellobiohydrolases (CBH)” or “exoglucanases” is an enzyme (EC 3.2.1.91) that hydrolyzes cellobiose from the reducing or non-reducing end of cellulose to degrade crystalline cellulose.

As used herein, a “β-glucosidase” or “β-D-glucoside glucohydrolase” is an enzyme (EC 3.2.1.21) that acts to liberate D-glucose units from cellobiose, cello-oligosaccharides, and other glucosides

As used herein, “hemicellulose” is a polymer component of plant materials that contains sugar monomers other than glucose, in contrast to cellulose, which contains only glucose. In addition to glucose, hemicellulose may include xylose, mannose, galactose, rhamnose, and arabinose, and the like, with xylose being the most common sugar monomer. Hemicelluloses contain most of the D-pentose sugars, and occasionally small amounts of L-sugars. The sugars in hemicellulose may be linked by ester linkages as well as glycosidic linkages. Exemplary forms of hemicellulose include but are not limited to are galactan, mannan, xylan, arabanan, arabinoxylan, glucomannan, galactomanan, and the like.

As used herein, the term “hemicellulase” refers to a class of enzymes capable of breaking hemicellulose into its component sugars or shorter polymers, and includes endo-acting hydrolases, exo-acting hydrolases, and various esterases.

As used herein, a “primary cellulase” or “primary celluloytic enzyme” is a cellulase that is required to efficiently hydrolyze cellulose or to produce glucose from a cellulosic substrate. Primary cellulases include CBH1, CBH2, EG1, EG2, and β-glucosidase.

As used herein, the term “accessory enzyme” refers to an enzyme that may be included in a cellulase composition to improve the efficiency of a cellulase (or combination of cellulases) but is not required for the efficient hydrolysis of cellulose or the production of glucose from a cellulosic substrate. Accessory enzymes include swollenin, EG4, cellulose induced protein (CIP1), and xylanase.

As used herein, a “naturally occurring” composition is one produced in nature or by an organism that occurs in nature.

As used herein, a “variant” protein differ from the “parent” protein from which it is derived by the substitution, deletion, or addition of a small number of amino acid residues, for example, 1, 2, 3, 4, 5, 6, 7, 8, 9, 10, 11, 12, 13, 14, 15, 16, 17, 18, 19, 20, or more amino acid residues. In some cases, the parent protein is a “wild-type,” “native,” or “naturally-occurring” polypeptides. Variant proteins may be described as having a certain percentage sequence identity with a parent protein, e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at even at least 99%, which can be determined using any suitable software program known in the art, for example those described in CURRENT PROTOCOLS IN MOLECULAR BIOLOGY (Ausubel et al. (eds) (1987) Supplement 30, section 7.7.18). Preferred programs include the VECTOR NTI ADVANCE™ 9.0 (Invitrogen Corp. Carlsbad, Calif.), GCG PILEUP program, FASTA (Pearson et al. (1988) Proc. Natl, Acad. Sci USA 85:2444-2448), and BLAST (BLAST Manual, Altschul et al., Nat'l. Cent. Biotechnol. Inf., Nat'l Lib. Med. (NCIB NLM NIH), Bethesda, Md., and Altschul et al. (1997) Nucleic Acids Res. 25:3389-3402). Another preferred alignment program is ALIGN Plus (Scientific and Educational Software, PA), preferably using default parameters. Another sequence software program that finds use is the TFASTA Data Searching Program available in the Sequence Software Package Version 6.0 (Genetics Computer Group, University of Wisconsin, Madison, Wis.).

The use of the singular includes the plural unless specifically stated otherwise, and the use of “or” means “and/or” unless state otherwise. The terms “comprise,” “comprising,” “comprises,” “include,” “including,” and “includes” are not intended to be limiting. The term “consisting essentially of” means that other components or steps may optionally be present but are not essential to produce or bring about a described effect.

All patents and publications, including all amino acid and nucleotide sequences disclosed within such patents and publications, referred to herein are expressly incorporated by reference.

The following abbreviations/acronyms have the following meanings unless otherwise specified:
° C. degrees Centigrade
BSA bovine serum albumin
CBD carbohydrate-binding domain
cDNA complementary DNA
CMC carboxymethyl cellulose
CMC carboxymethylcellulose
dH₂O or DI deionized water
dIH₂O deionized water, Milli-Q filtration
DNA deoxyribonucleic acid
ds or DS dry solids content
EDTA ethylenediaminetetraacetic acid
eq. equivalent
ETOH ethanol
g or gm gram
GA glucoamylase
Genencor Danisco US Inc, Genencor Division, Palo Alto, Calif., USA
H₂O water
HPLC high pressure/performance liquid chromatography
hr hour
IPTG isopropyl β-D-thiogalactoside
IU international unit
kDa kiloDalton
kg kilogram
L liter
M molar
mg milligram
min and ′ minute
mL and ml milliliter
mm millimeter
mM millimolar
MW molecular weight
N normal
PCS pretreated corn stover
PEG polyethyleneglycol
pl isoelectric point
pNPG p-nitrophenyl-α-D-glucopyranoside
RNA ribonucleic acid
RPM revolutions per minute
SDS-PAGE sodium dodecyl sulfate-polyacrylamide gel electrophoresis
sec and ″ second
sp./spp. species (singular/plural)
Spz. SPEZYME
U unit
UFC ultra-filtered concentrate
v/v volume/volume
w/v weight/volume
w/w weight/weight
wt % weight-percent
μg microgram
μL and μl microliter
μm micrometer
μM micromolar

### II. The Use of Swollenin to Enhancing the Sugar Yield from Cellulosic Feedstocks

An aspect of the compositions and methods relates to enhancing the sugar yield from cellulosic feedstocks/substrates by supplementing a cellulase composition with swollenin. In one aspect, a method for enhancing the enzymatic hydrolysis of cellulose is provided, the method comprising: (a) combining a cellulosic substrate, a mixed cellulase composition and swollenin; and (b) incubating the cellulosic substrate, mixed cellulase composition and swollenin under conditions conducive to hydrolyze cellulose. The compositions and methods are based on the surprising observation that replacement of up to 50% of a mixed cellulase composition with a swollenin can substantially enhance the amount of fermentable sugar generated from a cellulosic substrate.

A variety of proteins, called “expansins” have been identified in a variety of food plants. These expansins are believed to enhance osmotic uptake of water, which is the driving force of plant cell expansion. As water enters the cell, the protoplast expands but is restrained by the cell wall, which is held together by a rigid complex of cellulose microfibril polymers embedded in a glue-like matrix of pectins, hemicelluloses and proteins. The expansin family of proteins, found in various fruits, vegetables, grains, and oats, function as such a “wall loosening” factor, which alters the mechanical properties immature cell wall and allows it to undergo a process of elongation (see, e.g., Shcherban et al. (1995) Proc. Nat'l. Acad. Sci, U.S.A. 92:9245-49; Wang et al. (1994) Biotech. Lett. 16:955-58; Keller et al. (1995) The Plant Journal 8:795-802; Li et al. (1993) Planta, Vol. 191, pp. 349-56). Expansins play an important role in plant cell growth, fruit softening, abscission, emergence of root hairs, pollen tube invasion of the stigma and style, meristem function, and other developmental processes where cell wall loosening occurs.

More recently, expansin-like enzymes have been identified from microbial hosts. Once such enzyme, called swollenin, derived from Trichoderma reesei, is described in U.S. Pat. No. 6,458,928 (incorporated herein by reference). The sequence of swollenin partly resembles plant expansins, which are thought to break hydrogen bonds between polysaccharides in a cell wall. The native polypeptide sequence, including the N-terminal signal peptide, is shown as i.e., SEQ ID NO: 1. The mature polypeptide sequence is shown as SEQ ID NO: 2. Unlike expansins, mature swollenin includes at its N-terminus a cellulose-binding domain (CBD) that is linked via a linker region to an expansin-like domain. A CBD of this kind also occurs in well-known T. reesei cellulases, such as CBH I and EG II. Unlike swollenin cellulases are hydrolytic, at least to some extent.

(SEQ ID NO: 1)
MetAlaGlyLysLeuIleLeuValAlaLeuAlaSerLeuValSerLeu
SerIleGlnGlnAsnCysAlaAlaLeuPheGlyGlnCysGlyGlyIle
GlyTrpSerGlyThrThrCysCysValAlaGlyAlaGlnCysSerPhe
ValAsnAspTrpTyrSerGlnCysLeuAlaSerThrGlyGlyAsnPro
ProAsnGlyThrThrSerSerSerLeuValSerArgThrSerSerAla
SerSerSerValGlySerSerSerProGlyGlyAsnSerProThrGly
SerAlaSerThrTyrThrThrThrAspThrAlaThrValAlaProHis
SerGlnSerProTyrProSerIleAlaAlaSerSerCysGlySerTrp
ThrLeuValAspAsnValCysCysProSerTyrCysAlaAsnAspAsp
ThrSerGluSerCysSerGlyCysGlyThrCysThrThrProProSer
AlaAspCysLysSerGlyThrMetTyrProGluValHisHisValSer
SerAsnGluSerTrpHisTyrSerArgSerThrHisPheGlyLeuThr
SerGlyGlyAlaCysGlyPheGlyLeuTyrGlyLeuCysThrLysGly
SerValThrAlaSerTrpThrAspProMetLeuGlyAlaThrCysAsp
AlaPheCysThrAlaTyrProLeuLeuCysLysAspProThrGlyThr
ThrLeuArgGlyAsnPheAlaAlaProAsnGlyAspTyrTyrThrGln
PheTrpSerSerLeuProGlyAlaLeuAspAsnTyrLeuSerCysGly
GluCysIleGluLeuIleGlnThrLysProAspGlyThrAspTyrAla
ValGlyGluAlaGlyTyrThrAspProIleThrLeuGluIleValAsp
SerCysProCysSerAlaAsnSerLysTrpCysCysGlyProGlyAla
AspHisCysGlyGluIleAspPheLysTyrGlyCysProLeuProAla
AspSerIleHisLeuAspLeuSerAspIleAlaMetGlyArgLeuGln
GlyAsnGlySerLeuThrAsnGlyValIleProThrArgTyrArgArg
ValGlnCysProLysValGlyAsnAlaTyrIleTrpLeuArgAsnGly
GlyGlyProTyrTyrPheAlaLeuThrAlaValAsnThrAsnGlyPro
GlySerValThrLysIleGluIleLysGlyAlaAspThrAspAsnTrp
ValAlaLeuValHisAspProAsnTyrThrSerSerArgProGlnGlu
ArgTyrGlySerTrpValIleProGlnGlySerGlyProPheAsnLeu
ProValGlyIleArgLeuThrSerProThrGlyGluGlnIleValAsn
GluGlnAlaIleLysThrPheThrProProAlaThrGlyAspProAsn
PheTyrTyrIleAspIleGlyValGlnPheSerGlnAsn
(SEQ ID NO: 2)
GlnGlnAsnCysAlaAlaLeuPheGlyGlnCysGlyGlyIleGlyTrp
SerGlyThrThrCysCysValAlaGlyAlaGlnCysSerPheValAsn
AspTrpTyrSerGlnCysLeuAlaSerThrGlyGlyAsnProProAsn
GlyThrThrSerSerSerLeuValSerArgThrSerSerAlaSerSer
SerValGlySerSerSerProGlyGlyAsnSerProThrGlySerAla
SerThrTyrThrThrThrAspThrAlaThrValAlaProHisSerGln
SerProTyrProSerIleAlaAlaSerSerCysGlySerTrpThrLeu
ValAspAsnValCysCysProSerTyrCysAlaAsnAspAspThrSer
GluSerCysSerGlyCysGlyThrCysThrThrProProSerAlaAsp
CysLysSerGlyThrMetTyrProGluValHisHisValSerSerAsn
GluSerTrpHisTyrSerArgSerThrHisPheGlyLeuThrSerGly
GlyAlaCysGlyPheGlyLeuTyrGlyLeuCysThrLysGlySerVal
ThrAlaSerTrpThrAspProMetLeuGlyAlaThrCysAspAlaPhe
CysThrAlaTyrProLeuLeuCysLysAspProThrGlyThrThrLeu
ArgGlyAsnPheAlaAlaProAsnGlyAspTyrTyrThrGlnPheTrp
SerSerLeuProGlyAlaLeuAspAsnTyrLeuSerCysGlyGluCys
IleGluLeuIleGlnThrLysProAspGlyThrAspTyrAlaValGly
GluAlaGlyTyrThrAspProIleThrLeuGluIleValAspSerCys
ProCysSerAlaAsnSerLysTrpCysCysGlyProGlyAlaAspHis
CysGlyGluIleAspPheLysTyrGlyCysProLeuProAlaAspSer
IleHisLeuAspLeuSerAspIleAlaMetGlyArgLeuGlnGlyAsn
GlySerLeuThrAsnGlyValIleProThrArgTyrArgArgValGln
CysProLysValGlyAsnAlaTyrIleTrpLeuArgAsnGlyGlyGly
ProTyrTyrPheAlaLeuThrAlaValAsnThrAsnGlyProGlySer
ValThrLysIleGluIleLysGlyAlaAspThrAspAsnTrpValAla
LeuValHisAspProAsnTyrThrSerSerArgProGlnGluArgTyr
GlySerTrpValIleProGlnGlySerGlyProPheAsnLeuProVal
GlyIleArgLeuThrSerProThrGlyGluGlnIleValAsnGluGln
AlaIleLysThrPheThrProProAlaThrGlyAspProAsnPheTyr
TyrIleAspIleGlyValGlnPheSerGlnAsn

In some embodiments, the swollenin is a naturally-occurring variant T. reesei swollenin that has at least 80%, at least 85%, at least 90%, at least 91%, at least 92%, at least 93%, at least 94%, at least 95%, at least 96%, at least 97%, at least 98%, or even at least 99% amino acid sequence identity to an amino acid sequence of SEQ ID NO: 1. In some embodiments, the swollenin is obtained from a different organism, and has at least 80%, at least 85%, at least 90%, at least 91%, at least 92%, at least 93%, at least 94%, at least 95%, at least 96%, at least 97%, at least 98%, or even at least 99% amino acid sequence identity to an amino acid sequence of SEQ ID NO: 1.

In further embodiments, the swollenin is an engineered variant swollenin, that includes at least one substitution, insertion, or deletion that imparts an advantageous feature to the swollenin, and wherein the remainder of the amino acid sequence (i.e., not including the one or more substitutions, insertions, or deletions) has at least 80%, at least 85%, at least 90%, at least 91%, at least 92%, at least 93%, at least 94%, at least 95%, at least 96%, at least 97%, at least 98%, or even at least 99% amino acid sequence identity to an amino acid sequence of SEQ ID NO: 1. The substitution, insertion, or deletion may be in the N-terminal CBD, thereby affecting cellulose binding, or in a portion of the polypeptide other than the CBD, thereby affecting, e.g., the disruption of hydrogen bonds in a cellulosic substrate. In related embodiments, the swollenin is fragment or domain of swollenin that retain the biological activity described herein. In particular embodiments, the fragment lacks the CBD.

Suitable cellulosic substrates for use with swollenin include wood, wood pulp, papermaking sludge, paper pulp waste streams, particle board, corn stover, corn fiber, rice, paper and pulp processing waste, woody or herbaceous plants, grasses, rice hulls, cottonstraw, corn cobs, distillers grains, leaves, wheat straw, coconut hair, switchgrass, and mixtures thereof. An aspect of the present compositions and methods is the discovery that addition of swollenin to cellulase enhances sugar production in woody and herbaceous substrates. On the other hand, swollenin is not believed to be advantageous for use with grain-based or fruit-based substrates. Generally, due to swollenin's ability to interrupt hydrogen bonding, any substrate in which hydrogen bonding is prevalent (e.g., crystalline cellulose) are likely to be susceptible to swollenin activity, and are therefore suitable substrates. Exemplary cellulosic substrates have a high lignin content, as in the case of, e.g., softwoods. In some cases, the cellulosic substrate has a kappa number of 80 or higher, for example, 80, 81, 82, or higher.

The cellulosic substrate can be used directly (i.e., without pretreatment), or may be subjected to pretreatment using conventional methods that are known in the art. Exemplary pretreatments are chemical, physical, and biological pretreatments. Physical pretreatment techniques include, without limitation, various types of milling, crushing, steaming/steam explosion, irradiation, and hydrothermolysis. Chemical pretreatment techniques include, without limitation, dilute acid, alkaline, organic solvent, ammonia, sulfur dioxide, carbon dioxide, and pH-controlled hydrothermolysis. Biological pretreatment techniques include, without limitation, applying lignin-solubilizing microorganisms to the substrate.

The enzymatic hydrolysis of cellulose is preferably carried out at a temperature in a range of about 45° C. to about 75° C., and a pH of from about 3.5 to about 7.5. The initial concentration of cellulose in the hydrolysis reactor, prior to the start of hydrolysis, is preferably from about 4% (w/w) to about 15% (w/w). The combined dosage of all primary cellulase enzymes may be from about 5 to about 45 mg protein per gram cellulose. The hydrolysis may be carried out for a time period of from about 12 hours to about 200 hours. Preferably, the hydrolysis is carried out for a period of 15 hours to 100 hours. It should be appreciated that the reaction conditions are not meant to limit the invention in any manner and may be adjusted as desired by those of skill in the art.

The hydrolysis process preferably converts about 80% to about 100% of the cellulose to soluble sugars, or any range between. More preferably, the enzymatic hydrolysis process converts from about 90% to about 100% of the cellulose to soluble sugars, or even from about 98% to about 100% of the cellulose to soluble sugars.

The hydrolysis using the mixed cellulase composition and swollenin may be batch hydrolysis, continuous hydrolysis, or a combination thereof. The hydrolysis may be agitated, unmixed, or a combination thereof. The hydrolysis is typically carried out in a hydrolysis reactor. The primary cellulase and swollenin enzymes are added to the pretreated cellulosic feedstock (also referred to as the “substrate”) prior to, during, or after the addition of the substrate to the hydrolysis reactor. The cellulase and swollenin can be added simultaneously or sequentially to the hydrolysis. Over the extended period of hydrolysis additional cellulase and/or swollenin added to the partially digested cellulosic substrate.

Swollenin may be isolated from a microbial strain that produces it naturally, but preferably it is produced by a genetically modified organism, wherein a gene coding swollenin or an active fragment thereof, is operatively linked to a strong promoter for overexpressing the protein. The resultant gene construct (i.e., nucleic acid sequence), including at least part of the gene encoding swollenin, is used to transform a heterologous or homologous host cell, which is subsequently cultivated under conditions to express the desired protein is expressed. Recombinant protein expression methods are known in the art. Suitable host cells include, for instance, filamentous fungi, such as Trichoderma spp. or Aspergillus spp., and yeast. A preferred mode for preparing swollenin is via transformation of a Trichoderma spp. host cell with a DNA construct comprising at least a fragment of DNA encoding a portion or all of the swollenin that is functionally attached to a promoter. The transformed host cell is then grown under conditions so as to express the desired protein.

Swollenin is preferably produced as an extracellular protein that is secreted into culture medium, which may be used as a source of swollenin directly (e.g., as a broth), or used to further isolated and/or purified swollenin using methods known in the protein art. Alternatively, where swollenin is expressed as an intracellular protein, the cells are disrupted, followed by isolation and/or purification of the intracellular swollenin.

Where it is desired to obtain the swollenin protein in the absence of cellulolytic activity, it is useful to obtain, for example, a Trichoderma host cell strain which has had one or more cellulase genes deleted prior to introduction of a DNA construct or plasmid containing the DNA fragment encoding the swollenin. Such strains may be prepared by the method disclosed in U.S. Pat. No. 5,246,853 and WO 92/06209, which disclosures are hereby incorporated by reference. By expressing a swollenin in a host microorganism that is missing one or more cellulase genes, the identification and subsequent purification procedures are simplified. However, for use in the present invention, it is not necessary to completely exclude cellulolytic enzymes from the purified swollenin.

In particular embodiments, swollenin or a derivative, thereof, is recovered in active form from the host cell after growth in liquid media. The swollenin may include appropriate post-translational processing. The expressed swollenin can be recovered from the medium by conventional techniques including separations of the cells from the medium by centrifugation, filtration, and precipitation of the proteins in the supernatant or filtrate with a salt, for example, ammonium sulfate. Alternatively or additionally, chromatographic procedures such as ion exchange chromatography or affinity chromatography may be used. Antibodies (polyclonal or monoclonal) may be raised against purified swollenin, fragments of purified swollenin, or synthetic peptides corresponding to portions of swollenin.

In some embodiments, swollenin proteins are isolated or purified form other components with which it is naturally associated or other component of cells used to express the swollenin. A purified swollenin need not be devoid of all other components, but has a higher ratio of swollenin to extraneous proteins (and/or other components) than that found in the natural state, in culture medium, or in lysed cells. Purification can be accomplished by recognized separation techniques, such as ion exchange chromatography, affinity chromatography, hydrophobic separation, dialysis, protease treatment, ammonium sulfate or other alt precipitation, centrifugation, size exclusion chromatography, filtration, microfiltration, gel electrophoresis, or separation on a gradient to remove whole cells, cell debris, impurities, extraneous proteins (including enzymes) that are undesired in the final composition. It is further possible to then add components to a swollenin containing composition, which provide additional benefits, for example, activating agents, anti-inhibition agents, ions, compounds to control pH, or other enzymes, such as cellulase.

The amount of swollenin added to a hydrolytic mixture may vary in accordance with the biomass to be treated but is typically about 0.1 to about 30 mg/g of cellulose, preferably about 2 mg/g to about 20 mg/g of cellulose, and even about 5 mg/g to about 15 mg/g of cellulose. Alternatively, the amount of swollenin used in the present invention can be determined based on the total cellulosic substrate or total raw or pretreated biomass. The swollenin treatment is carried out at about 20 to about 80° C., preferably at about 30 to about 50° C., at a pH range of about 3 to about 10, preferably about 4 to about 6, for about 0.1 to about 24 hours, preferably about 2 to about 6 hours, at about 1% to about 30% solids (dry) loading, preferably about 15% to about 25%.

Typically, the enzymes will be used in a ratio of about 5:1 to about 1:5 cellulase:swollenin. More preferably, the enzymes are used in a ratio of about 2:1 to about 1:2 cellulase:swollenin. The relative ratio of enzyme can vary depending upon the type of cellulosic substrate. In some cases, swollenin represents an approximately equal amount of enzyme in a composition for treating a cellulosic material, as compared to the amount of cellulase in the composition. An approximately equal amount means that about 40-60% of the enzymes are swollenin, e.g., approximately 50%. A microcrystalline cellulosic substrate (i.e., softwood pulp) rich in hydrogen boding may require more swollenin than for an amorphous substrate (i.e., phosphoric acid swollen cellulose) with relatively lower degree of hydrogen bonding.

A mixed cellulase composition for use as described may have three synergistic cellulolytic activities: endo-1,4-β-D-glucanase, exo-1,4-β-glucosidase, and β-D-glucosidase activities. Each of these activities may be provided by one or more cellulase enzymes, which represent the primary cellulases (and activities, thereof) in the present compositions and methods. Any cellulase enzyme in the mixed cellulase composition can provide one or more of the three cellulolytic activities. Exemplary primary cellulases include CHB1, CBH2, EG1, EG2, and β-glucosidase. The cellulase composition may be an aqueous solution protein in water, a slurry of protein in water, a solid powder or granule, or a gel. A blend comprising cellulase enzymes may include additives, such as buffers, detergents, stabilizers, fillers, or other such additives familiar to those skilled in the art.

In some embodiments, the present compositions and method do not require an additional accessory enzyme (i.e., other than swollenin) in combination with a primary cellulase or combination of cellulases, as may be found in a whole cellulase broth. Such addition accessory enzymes include EG4, CIP1, and xylanase. Thus, in certain embodiments the present compositions and methods consist essentially of swollenin and one or more primary cellulases in the absence of accessory enzymes such as EG4, CIP1, and/or xylanase. In particular embodiments, the compositions and methods consist essentially of swollenin and one or more primary cellulases in the absence of EG4 or CIP1. In a more particular embodiment, the compositions and methods consist essentially of swollenin and one or more primary cellulases in the absence of EG4 and CIP1.

The cellulase and/or swollenin may be derived from microbial origins, and particularly from fungal or bacterial origins. Microorganisms that possess cellulolytic capabilities may be sources of both cellulase and swollenin proteins. In some embodiments, the cellulase and/or swollenin is derived from Trichoderma spp., particularly Trichoderma reesei (longibrachiatum). However, the cellulase and/or swollenin may also be derived from a fungus, such as Absidia spp.; Acremonium spp.; Agaricus spp.; Anaeromyces spp.; Aspergillus spp., including A. auculeatus, A. awamori, A. flavus, A. foetidus, A. fumaricus, A. fumigatus, A. nidulans, A. niger, A. oryzae, A. terreus and A. versicolor; Aeurobasidium spp.; Cepha/osporum spp.; Chaetomium spp.; Chrysosporium spp.; Coprinus spp.; Dactylium spp.; Fusarium spp., including F. conglomerans, F. decemcellulare, F. javanicum, E lini, Foxysporum and F. solani; Gliocladium spp.; Humicola spp., including H. insolens and H. lanuginosa; Mucor spp.; Neurospora spp., including N. crassa and N. sitophila; Neocaffimastix spp.; Orpinomyces spp.; Penicillium spp; Phanerochaete spp.; Phlebia spp.; Piromyces spp.; Pseudomonas spp.; Rhizopus spp.; Schizophyllum spp.; Trametes spp.; Trichoderma spp., including T. reesei, T. reesei (longibrachiatum) and T. viride; and Zygorhynchus spp. Similarly, it is envisioned that swollenin and/or DNA encoding swollenin may be found in cellulolytic bacteria such as Bacillus spp.; Cellulomonas spp.; Clostridium spp.; Myceliophthora spp.; Thermomonospora spp.; Streptomyces spp., including S. olivochromogenes; specifically fiber degrading ruminal bacteria such as Fibrobacter succinogenes; and in yeast including Candida torresii; C. parapsllosis; C. sake; C. zeylanoides; Pichia minuta; Rhodotorula glutinis; R. mucilaginosa; and Sporobolomyces ho/saticus.

Aspects of the present compositions and metthods may be further understood in light of the following examples, which should not be construed as limiting. Modifications to materials and methods will be apparent to those skilled in the art.

### EXAMPLES

The following examples are provided to illustrate the compositions and methods.

### Example 1

### Evaluation of Swollenin on Softwood Pulp

Softwood pulp equivalent to 0.1 g of cellulose was added to a 20 mL glass scintillation vial for each test. Each vial is brought to total volume of 10 mL, minus the amount of enzyme to be added in each test, by the additional of distilled water. The contents of each vial were brought to 50° C. by warming in the incubator set at 50°±1° C. To each vial 1 or 2 mg a whole cellulase composition obtained from Trichoderma (i.e., SPEZYME® CP, specific activity=3200-4110 IU/g; Genencor International, Inc., Palo Alto, Calif., USA,) was added to a final cellulase concentration of 10 mg cellulase per g cellulose or 20 mg cellulase per g cellulose (as described in Table 1). Purified swollenin or Bovine Serum Albumin (BSA, Sigma), as a control, was added to a final concentration of 10 mg/g cellulose (Table 1). Swollenin was prepared, purified and characterized according to the procedure described by Saloheimo et al. (2002) Eur. J. Biochem. 269:4202-11 (see Example 5, infra). The concentration of swollenin protein was estimated by gel electrophoresis and was approximately 3 mg/ml in both preparations.

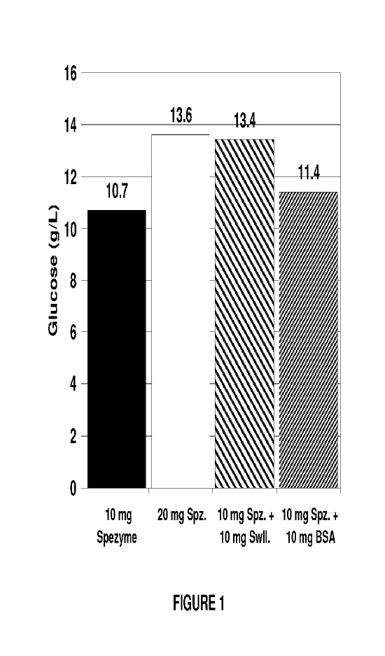

The vials were closed and incubated with gentle rotation (180 RPM) at 50° C. for a period of 24 hours. An aliquot was taken for analysis. Solids were removed by centrifugation. The supernatant was subjected to glucose analysis using either a YSI glucose analyzer or a Waters Alliance HPLC system. The results are shown in Table 1 and FIG. 1. Cellulase when supplemented with swollenin increased glucose concentration for both softwood and PCS substrates in 24 hr. Under the same total protein loading conditions (20 mg), 10 mg swollenin was essentially capable of replacing 10 mg celluase. The control protein (BSA) did not produce the same effect.

TABLE 1

| | Glucose (g/L) | |
|---|---|---|
| Enzyme (/g cellulose) | softwood | PCS 7.2% |
| 10 mg cellulase | 10.7 | 46 |
| 20 mg cellulase | 13.6 | 51.9 |
| 10 mg cellulase + 10 mg swollenin | 13.4 | 49 |
| 10 mg cellulase + 10 mg bovine serum albumin | 11.4 | 45.5 |

### Example 2

### Substrate Selectivity of Swollenin

Carboxymethyl cellulose (CMC), 1%; solka Floc (a synthetic, pure microcrystalline cellulose), 1%; softwood (kappa value=0); softwood (kappa value=82); mixed hardwood (kappa=81); softwood (kappa=80); and waste pulp (kappa=60); were tested for enhanced glucose production upon addition of swollenin. Each cellulosic substrate in an amount equivalent to 0.1 g of cellulose was added to a 20 mL glass scintillation vial for each test. 5.0 mL of a solution containing 0.1 M sodium citrate buffer (pH 4.8), 40 μL (400 μg) tetracycline, and 30 μL (300 μg) cycloheximide was added to each vial. Each vial was brought to total volume of 10 mL, minus the amount of enzyme to be added in each test, by the additional of distilled water. The contents of each vial were brought to 50° C. by warming in an incubator set at 50°±1° C. To each vial a whole cellulase composition obtained from Trichoderma (i.e., SPEZYME® CP, specific activity=3200-4110 IU/g; Genencor International, Inc., Palo Alto, Calif., USA,) was added to a final cellulase concentration of 20 mg cellulase per g cellulose. Additionally, β-glucosidase was added to a final concentration of 64 pNPG U/g cellulose.

Semi-purified swollenin was added to a final concentration of 10 mg/g cellulose (referring to Table 1). As before, swollenin was prepared, purified and characterized according to the procedure described by Saloheimo et al. (2002) Eur. J. Biochem. 269:4202-11 (see Example 5, infra). The concentration of swollenin protein was estimated by gel electrophoresis and was approximately 2 mg/ml in both preparations.

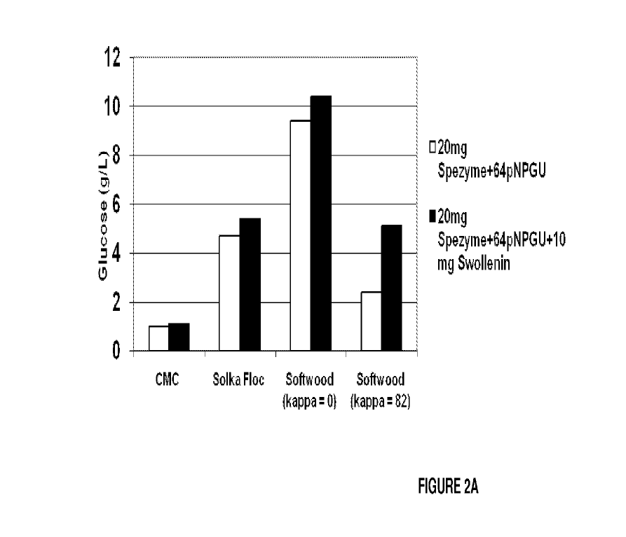

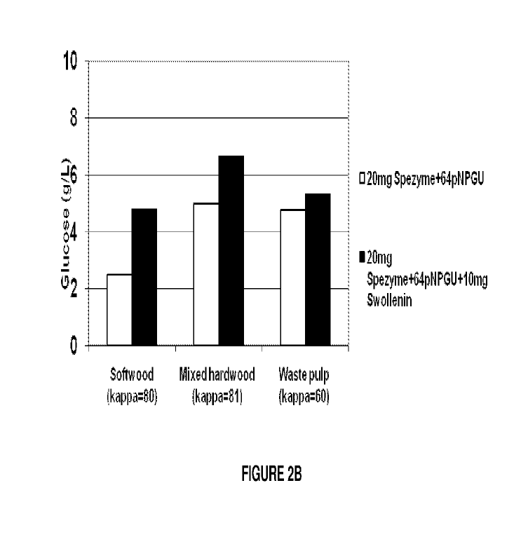

The vials were closed and incubated with gentle rotation (180 RPM) at 50° C. for a period of 24 hours. An aliquot was taken for analysis. Solids were removed by centrifugation. The supernatant was subjected to glucose analysis using a YSI glucose analyzer or a Waters Alliance HPLC system. The results are shown in Table 2 and in FIGS. 2A and 2B.

TABLE 2

| | | Glucose (g/L) | |
|---|---|---|---|
| Substrate | Kappa value | no swollenin | 10 mg/g swollenin |
| CMC | 0 | 1 | 1.1 |
| Solka Floka | 0 | 4.7 | 5.4 |
| softwood | 0 | 9.4 | 10.4 |
| | 82 | 2.4 | 5.1 |
| | 80 | 2.5 | 4.8 |
| hardwood | 81 | 5 | 6.65 |
| waste pulp | 60 | 4.75 | 5.3 |

Compared to synthetic substrate such as CMC or Solka Floka, a greater benefit from the presence swollenin was obtained using a cellulosic substrate such as softwood or hardwood pulp, as evidenced by the observation that more glucose sugar was liberated from the substrate. This effect is more pronounced in a high lignin substrate (e.g., softwood pulp, kappa value=82) than a zero lignin substrate (e.g., softwood pulp, kappa=0). Thus, the addition of swollenin may be more beneficial in the case of a highly recalcitrant substrate, e.g., where the substrate's pretreatment process has not been optimized, the lignin content is high, and/or the hydrogen bonds in the substrate are still intact and uninterrupted.

### Example 3

### Evaluation of Swollenin on the Hydrolytic Performance of Cellulase on Softwood Pulp

Unbleached softwood pulp with a kappa value of 82 was obtained from Smurft Facture (Biganos, FR). The unbleached pulp was derived through the Kraft process, washed and then air dried. Each sample was prepared to provide a final composition containing 4% cellulose (glucan) by dry weight in a 10 mL volume of reaction mixture. Enzymes were added to the sample substrate (as set forth in Table 2) in 20 mL scintillation vials to a total liquid volume of 10 mL, which included 50 mM sodium citrate buffer (pH 4.8) and antibiotics (tetracycline and cycloehexamide). A whole cellulase composition obtained from Trichoderma (i.e., SPEZYME® CP, Genencor International, Inc., Palo Alto, Calif., USA) was used as a source of cellulase in the enzyme hydrolysis experiments. The specific activity of SPEZYME CP is 3200-4110 IU/g.

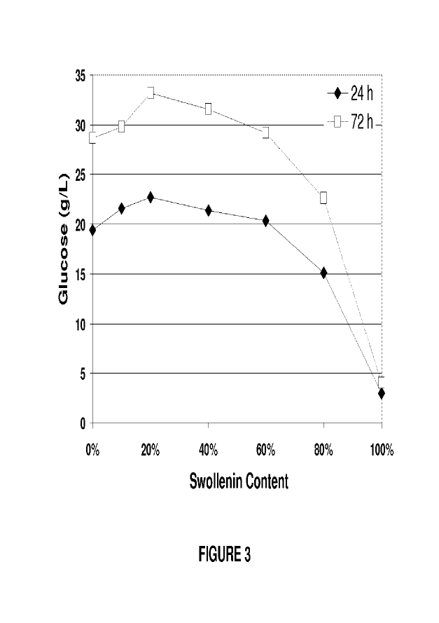

The suspensions were incubated with gentle shaking (180 rpm) at 50 C for 72 hours. Analysis of glucose content was performed after 24 and 72 hours following standard procedures. The results are shown in Table 3 and FIG. 3.

TABLE 3

| Cellulase | Swollenin | Swollenin | Glucose (g/L) | |
|---|---|---|---|---|
| (mg/g cellulose) | (mg/g cellulose) | Content | 24 h | 72 h |
| 30 | 0 | 0% | 19.4 | 28.6 |
| 27 | 3 | 10% | 21.6 | 29.8 |
| 24 | 6 | 20% | 22.7 | 33.2 |
| 18 | 12 | 40% | 21.4 | 31.5 |
| 12 | 18 | 60% | 20.3 | 29.2 |
| 6 | 24 | 80% | 15.1 | 22.6 |
| 0 | 30 | 100% | 3 | 4 |

### Example 4

### Swollenin Dosing Curve on Pulp and Paper Substrates

Two samples of unbleached softwood pulp were obtained from Smurft Facture (Biganos, FR). One (designated “high lignin”) had high lignin content (˜15%) and a kappa value (degree of lignification) of 82. The second (designated “low lignin”) had low lignin content (˜5%) and a kappa value (degree of lignification) of 12. The unbleached pulp was derived through Kraft process, washed and then air dried. Each sample was weighed out to obtain a final composition containing 4% of cellulose (glucan) by dry weight in a 10 mL volume of reaction mixture.

The softwood pulp used was weighed out so each sample contained exactly 0.4 g cellulose (glucan) by dry weight and dosed with as shown in Table 3. Enzymes were added to the sample substrate in 20 mL scintillation vial to a total liquid volume of 10 mL, which included 50 mM sodium citrate buffer (pH 4.8) and antibiotics (tetracycline and cycloehexamide). A cellulase enzyme complex produced from a genetically modified strain of Trichoderma reesei. (i.e., Accellerase 1000™, Genencor) was used as a source of cellulase in the enzyme hydrolysis experiments. Swollenin was used as a purified preparation, as above, and as described in Example 5. The concentration of swollenin was estimated to be approximately 2 mg/ml based on by gel electrophoresis. The purified extract is known not to have cellulase activity. The solid loading is 0.4 g cellulose in total liquid volume of 10 mL, giving a 4% cellulose (glucan) loading.

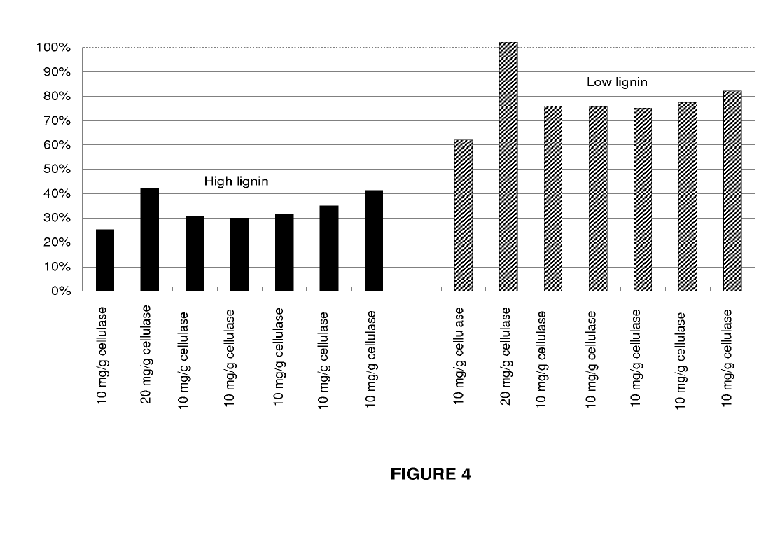

The reaction mixture was incubated with gentle shaking (200 rpm) at 50° C. for 72 hr. Analysis of glucose and cellobiose concentrations were performed using a Waters HPLC system (Alliance system, Waters Corp., Milford, Mass.). The HPLC column used for sugar analysis was purchased from BioRad (Aminex HPX-87H, BioRad Inc., Hercules, Calif.). Both glucose and cellobiose production were measured. The percent cellulose digestion (glucose generated plus cellobiose generated divided by input cellulose) is summarized in Table 4 and FIG. 4. Under the same total protein loading conditions (30 mg), swollenin was essentially capable of replacing 50% of the cellulase from Spezyme CP (Table 3). Supplementing ACCELLERASE with swollenin produced a beneficial effect using both high and low liginin substrates (Table 4).

TABLE 4

| Cellulase | Swollenin | % cellulose digestion | |
|---|---|---|---|
| (mg/g cellulose) | (mg/g cellulose) | High lignin | Low lignin |
| 10 | 0 | 10.6 | 25.4 |
| 20 | 0 | 17.8 | 41.6 |
| 10 | 5 | 13.1 | 30.2 |
| 10 | 10 | 13.2 | 29.7 |
| 10 | 15 | 13.6 | 29.2 |
| 10 | 20 | 14.8 | 30.0 |
| 10 | 30 | 17.1 | 31.5 |

### Example 5

### Purification of Swollenin

Buffer (50 mM Tris, pH 7.0) and sodium chloride (200 mM) were added to 2 L of the ultra-filtered concentrate (UFC) of a culture of a T. reesei strain in which the four major cellulases (CBH1, CBH2, EG1, and EG2) were deleted and the swollenin gene over-expressed under control of the cbhl promoter, while mixing. The pH was adjusted to pH 7.0. To this mixture was added 50 mL of a cellulose-binding domain (CDB) affinity purification agent (i.e., Cbind 200 resin, Part No. 70121, Novozymes A/S, Bagsvaerd, DK), followed by mixing for 1 hr. This mixture was then filtered using a scintered glass filter unit and the unbound material was collected. The resin with bound swollenin was washed twice using 2 L 50 mM Tris, pH 7.0 and 200 mM sodium chloride. Swollenin was eluted from the resin by mixing the resin with bound swollenin with MilliQ water (2 L) for 0.5 hr and then filtering the mixture, using the scintered glass filter unit, into a 2 L receiver. This water-elution process was repeated to make sure that the swollenin was thoroughly eluted.

The eluate was concentrated using a ultrafiltration system with a 10K MW cutoff. The concentrate (800 mL) was ready for analysis and further use. The protein concentration of swollenin was estimated to be approximately 3 mg/ml based on by gel electrophoresis. Note that the prepurified extract is known to have no significant cellulase activity.

1. A method for increasing the efficiency of cellulose hydrolysis using a cellulase, the method comprising:
(a) expressing a swollenin polypeptide using recombinant DNA technology,
(b) combining a cellulosic substrate, a cellulase composition, and the swollenin polypeptide, and
(c) incubating the cellulosic substrate, cellulase composition, and swollenin polypeptide under conditions conducive to hydrolysis of cellulose,
wherein the presence of the swollenin polypeptide increases the efficiency of cellulase hydrolysis when compared to the cellulase hydrolysis obtained using the cellulase composition in the absence of the swollenin polypeptide.

2. The method of claim 1, wherein the cellulase composition is a whole cellulase composition.

3. The method of claim 1, wherein the cellulase composition is a mixed cellulase composition.

4. The method of claim 1, wherein the cellulase composition comprises an endoglucanase, a cellobiohydrolases, and a β-glucosidase.

5. The method of claim 1, wherein the cellulase composition comprises one or more primary cellulases.

6. The method of claim 1, wherein the cellulase composition consists essentially of one or more primary cellulases.

7. The method of claim 5, wherein the primary cellulases are selected from CBH1, CBH2, EG1, EG2, and β-glucosidase.

8. The method of claim 1, performed in the absence of accessory enzymes other than swollenin.

9. The method of claim 1, performed in the absence of EG4 and CIP 1 .

10. The method of claim 1, performed in the absence of recombinant EG4 or recombinant CIP1.

11. The method of claim 1, wherein the ratio of cellulases in the celluase composition to swollenin (wt:wt) is between about 20:1 and about 1:5.

12. The method of claim 1, wherein the ratio of cellulases in the celluase composition to swollenin (wt:wt) is between about 10:1 and about 1:2.

13. The method of claim 1, wherein the ratio of cellulases in the celluase composition to swollenin (wt:wt) is between about 5:1 and about 1:1.5.

14. The method of claim 1, wherein the swollenin and the cellulases are present in an approximately equal amount (wt:wt).

15. The method of claim 1, wherein the cellulosic substrate is selected form the group consisting of wood, wood pulp, papermaking sludge, paper pulp waste streams, particle board, corn stover, corn fiber, rice, paper and pulp processing waste, woody or herbaceous plants, grasses, rice hulls, cottonstraw, corn cobs, distillers grains, leaves, wheat straw, coconut hair, switchgrass, and mixtures thereof.

16. The method of any claim 1, wherein the cellulosic substrate is a softwood.

17. The method of claim 1, wherein the cellulosic substrate is a high lignin substrate.

18. The method of claim 1, wherein the cellulosic substrate has a kappa number of 80 or higher.

19. The method of claim 1, wherein the percent increase in cellulase efficiency is at least about 10%.

20. An enzyme composition comprising:
(a) a mixed cellulase composition comprising an endoglucanase, a cellobiohydrolase, and a β-glucosidase, and
(b) a swollenin polypeptide expressed using recombinant DNA technology, wherein the ratio of swollenin as compared to the cellulases in said mixed cellulase is higher than the amount of swollenin in said mixed cellulase derived from a naturally occurring source of said mixed cellulases without said recombinant swollenin polypeptide.

21. The enzyme composition of claim 20, wherein the mixed cellulase composition does not include EG4 or CIP1.

22. The enzyme composition of claim 20, wherein the mixed cellulase composition is in an aqueous solution in water.

23. The enzyme composition of claim 20, wherein the mixed cellulase composition is in a slurry of protein in water.

24. The composition of claim 20, wherein the mixed cellulase composition consists essentially of primary cellulases.

25. An enzyme composition consisting essentially of:
(a) a mixed cellulase composition comprising an endoglucanase, a cellobiohydrolase, and a β-glucosidase, and
(b) a swollenin polypeptide expressed using recombinant DNA technology,
wherein the ratio of swollenin as compared to the cellulases in said mixed cellulase is higher than the amount of swollenin in said mixed cellulase derived from a naturally occurring source of said mixed cellulases without said recombinant swollenin polypeptide.

26. The composition of claim 20, wherein the ratio of cellulases in the mixed cellulase composition to swollenin (wt:wt) is between about 20:1 and about 1:5.

27. The composition of claim 20, wherein the ratio of cellulases in the mixed cellulase composition to swollenin (wt:wt) is between about 10:1 and about 1:2.

28. The composition of claim 20, wherein the ratio of cellulases in the mixed cellulase composition to swollenin (wt:wt) is between about 5:1 and about 1:1.5.

29. The composition of claim 20, wherein the swollenin and the cellulases are present in an approximately equal amount (wt:wt).

30. The composition of claim 20, wherein the amount of swollenin (wt:wt) in the composition replaces an approximately equal amount of cellulases (wt:wt) in the composition, with respect to cellulase efficiency on a cellulosic substrate.
